(19) United States
(12) Patent Application Publication
(10) Pub. No.: US 2021/0147900 A1
(43) Pub. Date: May 20, 2021

(54) METHODS AND COMPOSITIONS FOR DETECTING BETA-HYDROXYBUTYRATE IN BIOLOGICAL FLUIDS

(71) Applicant: F.A.T. Stats LLC, East Norriton, PA (US)

(72) Inventors: Tom Meitz, Audobon, PA (US); Frank Puglisi, East Norriton, PA (US); Joseph Rokowski, East Norriton, PA (US)

(21) Appl. No.: 16/951,050

(22) Filed: Nov. 18, 2020

Related U.S. Application Data
(60) Provisional application No. 62/936,946, filed on Nov. 18, 2019.

(51) Int. Cl. C12Q 1/32; G01N 21/78; G01N 33/52

(52) U.S. Cl. CPC C12Q 1/32; G01N 33/523; G01N 21/78

(57) ABSTRACT

Compositions and methods for detecting β-hydroxybutyrate in a biological fluid from a subject in need thereof are disclosed. Also disclosed are compositions and methods of reducing an optical change in a composition for detecting the β-hydroxybutyrate during storage before the composition is exposed to the β-hydroxybutyrate.

### CROSS-REFERENCE TO RELATED APPLICATIONS

This application claims the benefit of priority under 35 U.S.C. § 119(e) to earlier filed U.S. Provisional Patent Application No. 62/936,946 filed Nov. 18, 2019, the entire disclosure of which is incorporated by reference herein.

### BACKGROUND OF THE INVENTION

The invention relates to compositions useful for detecting β-hydroxybutyrate in a biological fluid, particularly urine. The invention also relates to methods of using the compositions to detect β-hydroxybutyrate in a biological fluid by way of detecting a change in an optical property of the composition when exposed to β-hydroxybutyrate in the biological fluid. Certain of the inventive compositions and methods relate to reducing a change in the optical property of the compositions in storage, prior to exposure to β-hydroxybutyrate in the biological fluid.

The underlying principles of the ketogenic diet (KD) focus on transitioning a diet to a high fat, moderate protein, and low carbohydrate macronutrient intake. The goal of this style of eating is to transition the body into a state of ketosis, in order to lose weight. Transitioning the body into a state of ketosis can also be beneficial in the management of hyperglycemia states. During ketosis the body utilizes fat as its primary source of energy in place of glucose (sugar). As this transition is taking place, hepatic ketone production begins.

The first ketone produced during hepatic ketone production is acetoacetate which can be measured in the urine using a dipstick test. Production of acetoacetate is short lived and therefore detection of it is an unreliable method to determine accurately if a subject is in a state of nutritional ketosis or diabetic ketoacidosis (DKA). As the body moves towards nutritional ketosis and the acetoacetate levels decrease, hepatic production of β-hydroxybutyrate (BHB) correspondingly increases. BHB is the primary ketone used by the body for energy once nutritional ketosis has been achieved. Currently, the only methods available to test BHB is via finger prick (capillary blood test) or whole blood serum testing. These methods are not only invasive but also very expensive to the consumer. These two factors often lead to decreased BHB testing and greatly increase the likelihood that the consumer will be unsuccessful with achieving/maintaining nutritional DKA.

Uncontrolled Type 1 diabetes and occasionally Type 2 diabetes can progress to diabetic ketoacidosis, due to inadequate insulin therapy management. As a result, the body responds with hepatic ketone production. Similar to nutritional ketosis, the initial ketone produced in this process is acetoacetate, which is quickly followed by production of BHB.

Accurate measurements of specific levels of BHB are indicative of certain physiological conditions in the human diet that reflect a high metabolic rate of fat burning and are useful to dieters and physical fitness enthusiasts. Because dietary input varies through the course of a day, it is useful to know quickly and accurately the fat burning rate and thus the level of BHB using a quick, colorimetric test strip that can test the urine of a subject.

Presently, a clinical diagnosis of diabetic ketoacidosis is determined by the combination of a urine dipstick test for acetoacetate, a test of blood level of serum BHB levels and a test for blood serum glucose level. This method of testing is not only expensive, but cumbersome and inefficient in the hospital or medical office setting. It has been shown that as diabetic ketoacidosis progresses, serum levels of acetoacetate begin to decrease while serum BHB levels increase. In turn, urinary BHB levels will also begin to increase. Currently, there is no commercially available easy-to-use diagnostic test for measuring human β-hydroxybutyrate in biological fluids, for example, urine. Current diabetic ketoacidosis management protocols include hydration to stabilize electrolytes and blood glucose levels. However, hydration decreases measurable blood serum BHB levels. This creates a false negative in regards to blood serum BHB levels and gives the clinical impression that the disease process is improving.

Current commercial tests claim accurate BHB analysis but only test for acetoacetate which is a byproduct of BHB degradation and do not test for BHB itself, and therefore such tests tend to be inaccurate in their diagnostic ability to specifically measure BHB.

U.S. Pat. No. 6,762,035 B1 discloses a diagnostic test for BHB in urine samples but the test described therein requires high levels of the enzyme β-hydroxybutyrate dehydrogenase (BHBD) for effective visual color indication of the presence of BHB. In addition, the enzymes used in the test disclosed in U.S. Pat. No. 6,762,035 B1 may not be stable and the test strips/kits with the enzymes may require storage under carefully controlled conditions in order to retain their effectiveness for BHB detection in urine samples. If not stored properly, the compositions used for colorimetric analysis in U.S. Pat. No. 6,762,035 B1 have been shown herein to change their optical properties, e.g., visual color, even at ambient temperature, in the absence of BHB and therefore render the test strips with the compositions disclosed therein inaccurate or non-useable for BHB testing.

Others have attempted to develop BHB diagnostic tests for urine samples. One of these, disclosed in European Patent Application Publication No. EP 2636750 A1 may require the use of a portable spectrophotometric device to measure a color change in conjunction with test indicators. Portable spectrophotometric devices to measure color change in test strips are cumbersome, expensive and require temperature sensitive reagents needed to be stored at low temperatures to run the test.

Accordingly, there remains a need for a composition useful for simple, accurate and non-invasive detection of BHB in a biological fluid such as urine from a subject that are suitable after prolonged storage prior to exposure to BHB.

### SUMMARY OF THE INVENTION

The present invention provides compositions and methods for detecting β-hydroxybutyrate (BHB) in a biological fluid such as urine based on the inventors' surprising discovery of a change in an optical property of the compositions comprising a redox cofactor such as 2,3-dimethoxy-5-methyl-p-benzoquinone (coenzyme Q₀), and reduction of the change by adding an inhibitor to the compositions comprising a redox cofactor such as coenzyme Q₀ or nicotinamide dinucleotide (NAD).

According to a first aspect of the invention, a composition for detecting β-hydroxybutyrate (BHB) is disclosed. The composition has an optical property that changes upon exposure to BHB. The composition comprises (a) nitro tetrazolium blue (NZT) or 2,3,5-triphenyltetrazolium chloride; (b) β-hydroxybutyrate dehydrogenase (BHBD); (c) diaphorase; and (d) a redox cofactor, wherein the redox cofactor is 2,3-dimethoxy-5-methyl-p-benzoquinone (coenzyme Q₀).

According to a second aspect of the invention, a composition for detecting β-hydroxybutyrate (BHB) is disclosed. The composition has an optical property that changes upon exposure to BHB and the composition comprises: (a) nitro tetrazolium blue (NZT) or 2,3,5-triphenyltetrazolium chloride; (b) β-hydroxybutyrate dehydrogenase (BHBD); (c) diaphorase; (d) a redox cofactor, which is 2,3-dimethoxy-5-methyl-p-benzoquinone (coenzyme Q₀) or nicotinamide dinucleotide (NAD); and (e) an inhibitor in an amount effective for reducing a change in the optical property of the composition for at least 6 hours before the composition is exposed to the BHB.

The redox cofactor may be coenzyme Q₀ and the inhibitor may be selected from the group consisting of nanoparticulate anatase TiO₂, nanoparticulate ZnO, nanoparticulate silica, nanoparticulate CaCO₃, nanoparticulate ZrO₂, NaNO₂, Ca(NO₃)₂, hydroxyectoine, and calcium nitrate. In one embodiment, the redox cofactor may be coenzyme Q₀ and the inhibitor may be nanoparticulate anatase TiO₂.

The redox cofactor may be NAD and the inhibitor may be nanoparticulate ZnO, nanoparticulate ZrO₂, NaNO₂, Ca(NO₃)₂, or trehalose.

The redox cofactor may be coenzyme Q₀ and the BHB may be in an aqueous sample.

The composition may further comprise cyclodextrin in an amount effective for solubilizing the coenzyme Q₀ in the aqueous sample. The aqueous sample containing BHB may be a biological fluid. The biological fluid containing BHB may be urine.

In some embodiments, the composition for detecting BHB may have a water content less than 0.3 wt %. The composition for detecting BHB may further comprise an effective amount of a buffer for maintaining a pH above 8.5 when the composition is exposed to an aqueous sample. The composition for detecting BHB may be on a carrier. The carrier may comprise a porous material. The carrier may further comprise an inert water-resistant substrate attached to the porous material.

According to a third aspect of the invention, a method of preparing a composition for detecting β-hydroxybutyrate (BHB) is disclosed. The method comprises the following steps:

(a) A step of: mixing nitro tetrazolium blue (NZT) or 2,3,5-triphenyltetrazolium chloride; β-hydroxybutyrate dehydrogenase (BHBD); diaphorase; and a redox cofactor to make a mixture. The redox cofactor is 2,3-dimethoxy-5-methyl-p-benzoquinone (coenzyme Q₀) or nicotinamide dinucleotide (NAD); and

(b) A step of: adding to the mixture an inhibitor to make a composition having an optical property that changes upon exposure to BHB, such that a change in the optical property of the composition is reduced for at least 6 hours before the composition is exposed to the BHB.

According to a fourth aspect of the invention, a method for detecting β-hydroxybutyrate (BHB) in a biological fluid from a subject is disclosed. The detection method may comprise the following steps:

(a) A step of exposing the biological fluid to a composition of the present invention such that an optical property of the composition is changed;

(b) A step of detecting the change of the optical property in step (a) such that the detected change indicates the presence of BHB in the biological fluid.

The method of detecting BHB may further comprise a step of storing the composition for at least at least 24 hours before performing step (a). In one embodiment, the biological fluid containing the BHB is urine. In another embodiment, the BHB is present in the biological fluid in an amount of at least 0.2 mM. In yet another embodiment, the BHB is present in the biological fluid over a range of 0 to 4 mM.

### DETAILED DESCRIPTION OF THE INVENTION

As disclosed herein, the present invention provides a composition for detecting β-hydroxybutyrate (BHB) and preparation thereof. The inventors have surprisingly discovered the use of coenzyme Q₀ (ubiquinone-0 or 2,3-dimethoxy-5-methyl-1,4-benzoquinone) in a composition for detecting BHB in a biological fluid, for example, urine, based on a change in an optical property of the composition upon exposure to the biological fluid, and the use of an inhibitor for reducing a change in the optical property of a composition for BHB detection comprising coenzyme Q₀ or nicotinamide adenine dinucleotide (NAD) during storage before the BHB detection.

This invention overcomes the aforementioned obstacles and offers a non-invasive and simple-to-use test to detect BHB levels in urine that may correspond to blood serum BHB levels in the same subject. The invention enables a subject to conveniently monitor his/her BHB level and successfully achieve and maintain nutritional ketosis while eating a ketogenic diet.

An invention in which urinary BHB is detected has clinical value. The invention may allow for hospital, physician office, and emergency situation testing for the presence of BHB in urine and also may be suitable for in-home self-monitoring by subjects in need of such testing. This method of testing may be non-invasive and may allow for accurate early detection of diabetic ketoacidosis. Early detection of diabetic ketoacidosis and subsequent treatment with hydration and insulin therapy may decrease overall mortality and comorbidity as the disease process of diabetic ketoacidosis progresses.

The present invention may improve the clinician's ability to more accurately reduce false negatives related to whole blood serum BHB levels after the initial hydration therapy has begun and evaluate the true progression of diabetic ketoacidosis in a subject in need of such monitoring. As urinary BHB levels of the subject decrease with treatment, the composition and methods according to the present invention for detecting BHB in urine may reflect the true improvement of the disease process compared to the current testing protocols which may comprise testing of whole blood serum for BHB levels and/or testing of urine for acetoacetate.

Generally, the invention provides a composition that undergoes a change in its optical property upon exposure to BHB. Such a change in an optical property may be detected using any conventional technique known in the art. The optical property may be observed as a visual color change, e.g., from a light color to a darker color, or from a nearly white color to a purple color. The optical property may also be measured as ΔE, by the use of a color meter, also referred to as a spectrophotometer. The composition for detecting BHB may further comprise an inhibitor to reduce undesirable change in the optical property (e.g., a visual color change and/or a change in ΔE) during storage before BHB detection.

A composition for detecting β-hydroxybutyrate (BHB) is provided. The composition comprises: (a) an indicator reagent; (b) β-hydroxybutyrate dehydrogenase (BHBD); (c) diaphorase; and (d) a redox cofactor. The composition has an optical property that changes upon exposure to BHB.

Indicator reagent: The indicator reagent according to the present invention may be any reagent known in the art that undergoes a change in its optical property upon reduction. Non-limiting examples of such compounds are tetrazolium compounds or salts thereof. Non-limiting examples of indicator reagents include nitro tetrazolium blue, triphenyl tetrazolium blue, tetranitroblue tetrazolium, resazurin, and 5-bromo-4-chloro-3-indolyl phosphate (TNBT).

The indicator reagent is present in an effective amount for detecting BHB. The effective amount of the indicator reagent may be, as weight percent of the total dried weight of the composition for detecting BHB, 0.5-10 wt %, 2-9 wt %, 3-8 wt %, 4-7 wt %, 5-6 wt %, 1-6 wt %, 2-7 wt %, or 3-6 wt %.

β-hydroxybutyrate dehydrogenase (BHBD): The source of the BHBD according to the present invention is not particularly limited. For example, the BHBD may be sourced from Alcaligenes or Pseudomonas strains of bacteria, or any other suitable bacteria, mutant sources, or animal source as known in the art.

The BHBD is present in an effective amount for detecting BHB. The effective amount of the BHBD may depend on the choice and/or amount of the redox cofactor. The effective amount of the BHBD in the dry composition may be, as weight percent of the total dried weight of the composition for detecting BHB, 0.07-2.3 wt %, 0.1-2.2 wt %, 0.5-2.1 wt %, 7-2, wt %, 1-2.3 wt %, 1-2 wt %, or 1.5-2 wt %.

Diaphorase: The source of the diaphorase according to the present invention is not particularly limited. For example, the diaphorase may be sourced from Clostridium kluyveri or Bacillus stearothermophilus, or any other suitable bacteria as known in the art.

The diaphorase is present in an effective amount for detecting BHB. The effective amount of the diaphorase may be, in weight percent of the dry weight of the dry composition for detecting BHB, 1.5-3 wt %, 1-2 wt % or 1.75-2.5 wt %.

Redox cofactors: The redox cofactor according to the present invention may be nicotinamide adenine dinucleotide (NAD), coenzyme Q₀ or a combination thereof.
NAD: The NAD is present in an effective amount for detecting BHB. The effective amount of the NAD may be, in weight percent of the dry weight of the dry composition for detecting BHB, 10-90 wt %. For example, the effective amount of the NAD may be 50-90 wt %, 60-80 wt %, 60-70 wt % or 85-90 wt %. In an embodiment, nicotinamide adenine dinucleotide phosphate hydrogen (NADPH) may be used.
Coenzyme Q₀: The coenzyme Q₀ is present in an effective amount for detecting BHB. The effective amount of the coenzyme Q₀ may be, in weight percent of the dry weight of the dry composition for detecting BHB, 0.01-40 wt %. For example, the effective amount of the coenzyme Q₀ may be, in weight percent of the dry weight of the dry composition for detecting BHB, 0.01-1 wt %, 0.01-0.5 wt %, 10-30 wt %, 1-25 wt %, or 15-30 wt %.

Coenzyme Q₀ in water with cyclodextrin: Coenzyme Q₀ is not soluble in water or may have limited solubility in water or an aqueous solution, for example, urine, under ambient conditions. This means that until now, coenzyme Q₀ could not feasibly be used as a redox cofactor in an aqueous solution, such a urine. However, the inventors have discovered that the addition of cyclodextrin to the composition for detecting BHB as disclosed herein improves solubility of the coenzyme Q₀ in the composition when exposed to a biological fluid such as urine. The term “cyclodextrin” as used herein refers to α-cyclodextrin (6 glucose subunits), β-cyclodextrin (7 glucose subunits), γ-cyclodextrin (8 glucose subunits), a derivative of any of these three types, or a combination thereof. The cyclodextrin may be present in an effective amount for solubilizing the coenzyme Q₀ in the composition for detecting BHB. The weight ratio of the cyclodextrin to the coenzyme Q₀ may be from 100:1 to 1:100, from 1:50 to 50:1, from 10:1 to 1:10, from 2:9 to 9:2, from 3:8 to 8:2, from 7:1 to 1:7, or from 2:1 to 1:1. Further, the effective amount of the cyclodextrin, in weight percent of the dry weight of the composition for detecting BHB may be 0.1-10 wt %, 1-5 wt %, or 3-4 wt %. The molar ratio of the cyclodextrin to the coenzyme Q₀ may be from 100:1 to 1:100. Non-limiting examples of derivatives of cyclodextrin include: β-cyclodextrin(-OH)₁₉(—ONO₂)₂, β-cyclodextrin(-OH)_{19.2}(—OPO₃H)_{1.8}, β-cyclodextrin(-OH)₁₉(—OSO₃H)₂, β-CD(-OH)_{18.5}(—O—CH₂—CO₂H)_{2.5}, β-cyclodextrin(-OH)_{19.3}(—O—CH₂CH₂CH₂—SO₃H)_{1.7}, β-cyclodextrin(-OH)_{18.5}(—O—CH₂CH₂CH₂—SO₃H)_{2.5}, β-cyclodextrin(-OH)_{18.0}(—O—CH₂CH₂CH₂—SO₃H)_{3.0}, β-cyclodextrin(-OH)₇(—OCH₃)₁₄, β-cyclodextrin(-OCH₃)₂₁, and mixtures thereof.

The amount of the cyclodextrin in the composition may be adjusted as needed, based on the amount of coenzyme Q₀ in the composition for detecting BHB.

Optical property: An optical property as referred to herein refers to an optical property of the composition for detecting BHB. The optical property of the composition for detecting BHB may be due to an optical property of the indicator reagent which can be optically detected. Non-limiting examples of such optical properties are light absorption or emission, re-emission, refraction or polarization and properties associated therewith. It will be understood that a change of at least one optical property as used herein, may encompass the detection of the presence of a property which was not detectable before, the detection of the absence of a property which has been detected before and the detection of quantitative changes of a property, i.e. the detection of the change of the signal strength which correlates to the extent of the change of the at least optical property. Optical properties contemplated by the present invention may be color, including color that can be detected visually, or by the use of a color meter that utilizes the CIELAB color space (also referred to as CIE L*a*b*), as defined by the International Commission on Illumination. Optical properties may be detectable spectrophotometrically, for example. Other non-limiting examples of optical properties may be fluorescence, luminescence, or refractometry, for example, a change in refractive index. The optical properties which may change or may be observed according to the present invention may depend on the type and level of the indicator reagent. A change in color, as for instance, from a yellow or white color to a purple color that can be detected visually is contemplated. A change in ΔE measured with a color meter is contemplated.

Theoretical General Reactions:

Without being bound by any theory, this invention may involve some general reactions.

An indicator reagent may be used for detecting β-hydroxybutyrate (BHB) in a biological fluid. The indicator reagent may have a first optical property in its unreduced form, but upon reduction, may change to a reduced form which may have a different, second optical property. Generally, this overall reaction may be done with two enzymes and a redox cofactor that together, may transfer a hydride (H—) from the BHB to the indicator reagent, thereby reducing the indicator reagent and causing the change in the optical property. A non-limiting example of a suitable indicator reagent is a tetrazolium compound such as nitro tetrazolium blue (NZT). According the present invention, at least one of two different redox coagents (also referred to as redox cofactors) may be used. Reaction Scheme 1, below, shows a theoretical series of reactions that may occur when using NAD (nicotinamide adenine dinucleotide) as the redox coagent.

Reaction scheme 1, below, utilizes NAD as a redox cofactor.

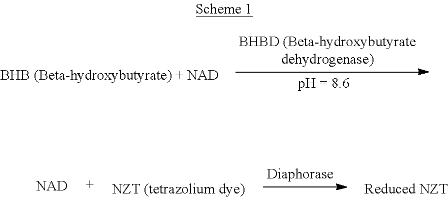

Reaction scheme 2, below, is similar to reaction scheme 1, but uses coenzyme Q₀ (2,3-dimethoxy-5-methyl-p-benzoquinone) as the redox coagent. Note that in the scheme below, the coenzyme Q₀ is referred to as CoQzero.

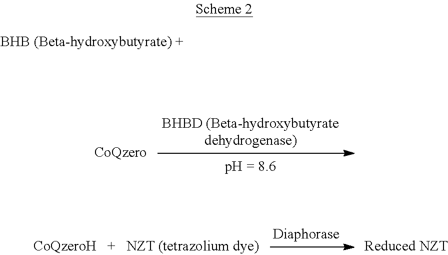

In both of these reaction schemes, the first enzyme, β-hydroxybutyrate dehydrogenase (BHBD) may move a hydride from the BHB to the redox coagent, e.g., the NAD or the CoQzero, thus forming NADH or CoQzeroH. The diaphorase enzyme may then transfer the hydride from the NADH or the CoQzeroH to the NZT indicator reagent, forming reduced NZT (NZTH), which may have a different optical property than the unreduced NZT. In particular, visually, the NZT may be colorless and the NZTH (i.e., reduced NZT) may be purple. This change in optical property or color may be measured with a color meter, or via spectrophotometry for example and may be expressed as ΔE. The reference value for the ΔE measurement, as used herein, is understood to be the color of a composition for detecting BHB comprising coenzyme Q₀ or NAD, BHBD, NZT, and diaphorase prior to exposure to the BHB.

This composition may further comprise an inhibitor in an amount effective for reducing a change in the optical property of the composition before the composition is exposed to BHB. The inhibitor may be effective for a predetermined time period, for example, from 1 hour to 5 years, from 6 months to 5 years, from 3 months to 3 years, from 1 month to 2 years, from 2 years to 3 years, from 1 hour to 90 days, from 24 hours to 60 days, from 2 days to 60 days, from 48 hours to 30 days, from 1 hour to 6 hours, from 6 hours to 24 hours, from 1 hour to 48 hours, from 6 hours to 72 hours, from 1 hour to 72 hours, from 12 hours to 1 week, from 2 hours to 48 hours, from 30 days to 90 days, from 60 days to 120 days, or from 6 hours to 2 weeks.

Inhibitors: Suitable inhibitors for reducing a change in the optical property may be selected from the following: nanoparticulate anatase TiO₂, nanoparticulate rutile TiO₂, nanoparticulate ZnO, nanoparticulate silica, nanoparticulate CaCO₃, nanoparticulate ZrO₂, nanoparticulate MgO, NaNO₂, Ca(NO₃)₂, hydroxyectoine, calcium nitrate, trehalose, 2-O-alpha-mannosyl-D-glycerate, barium chloride, magnesium chloride, bovine serum albumen, polyethylenimine, polypropylenimine, polyfunctional azridines, glucosylglycerol, glucosylglycerate, diglycerol phosphate, N-γ-acetyldiaminobutyrate, α-hydroxyectoine, ectoine, dithiothreitol, mercaptoethanol, 5× Antigen-Down Conjugate Stabilizer (CS2), alkaline phosphatase, choline chloride, choline acetate, boronic acids such as nitrophenylboronic acid, borates, sorbitol, sucrose, carboxylic acid salts (e.g., sodium citrate, sodium succinate, sodium tartarate, sodium malonate, sodium gluconate), polyethylene glycol, and a combination thereof. The term “nanoparticulate” as used herein refers to particles having a number average diameter in the range from 1 nm to 100 nm, as measured using conventional technique known in the art, for example, laser scattering.

The inhibitor is present in an effective amount for reducing a change in the optical property of the composition. The effective amount of the inhibitor may range from 0.0001 wt % to 50 wt %, based on the dry weight of the composition for detecting BHB. For example, the effective amount of the inhibitor may be 0.0001-25 wt %, 0.0001-15 wt %, 0.0001-10 wt %, 0.0001-5 wt %, 0.001-0.1 wt %, 0.5-10 wt %, 4-6 wt %, or 0.001-0.05 wt %, based on the dry weight of the composition.

The composition may be in the form of an aqueous solution or suspension. The composition may be on a carrier and then optionally dried, so as to form, for example, a test strip, or a test kit. The carrier may be a porous material, such as filter paper. The carrier material with the composition may be attached to a water-resistant substrate for ease of handling as well. The composition may optionally comprise a buffer at a level for maintaining the composition at a desirable pH when the composition is exposed to an aqueous solution or suspension, for example, urine.

Where the redox cofactor is coenzyme Q₀, the composition may further comprise cyclodextrin. The coenzyme Q₀ may be in amount sufficient to solubilize the coenzyme Q₀ in an aqueous solution or suspension, for example, urine.

In one embodiment, the composition has an optical property that changes upon exposure to BHB. The composition comprises the following: (a) an indicator reagent which may be nitro tetrazolium blue (NZT) or 2,3,5-triphenyltetrazolium chloride; (b) β-hydroxybutyrate dehydrogenase (BHBD); (c) diaphorase; (d) a redox cofactor, which may be 2,3-dimethoxy-5-methyl-p-benzoquinone (coenzyme Q₀) or nicotinamide dinucleotide (NAD); and (e) an inhibitor in an amount effective for reducing a change in the optical property of the composition during storage.

Storage Conditions:

The composition for detecting BHB may be stored at a predetermined temperature and/or a predetermined relative humidity for a predetermined time period. For example, the storage temperature may be between 20° C. and 30° C. The storage relative humidity may be 40% to 50%. The predetermined time period may be up to 90 days, three months, one year, 2 years, 3 years or 5 years prior to use for BHB detection. The composition for detecting BHB may be stored at a temperature of 10° C., 11°, 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C. The composition for detecting BHB may be stored at a temperature between any two of these temperatures, for example, between 10° C. and 40° C. or between 20° C. and 35° C. The range of relative humidity may be from 5-40%, from 10-30%, or from 10-20%.

Further, the composition for detecting BHB may be stored in the dark. The term “dark” used herein refers to light intensity less than 0.001 lux. For example, the composition for detecting BHB may be stored in a thick, lidded, light impervious cardboard box lined with aluminum foil inside and covered with aluminum foil on the outside to reduce light intensity within the box to be less than 0.0001 lux. The box may then be stored in a BINDER environmental chamber at 50% relative humidity and 22° C.

The composition for detecting BHB may be stored in the dark at between 10° C. and 40° C. for a period of up to 2 hours, up to 6 hours, up to 12 hours, up to 24 hours, up to 36 hours, up to 48 hours, up to 60 hours, up to 72 hours, up to 96 hours, up to 5 days, up to 6 days, up to 1 week, up to 2 weeks, up to 3 weeks, up to 1 month, up to 2 months, up to 3 months, up to 1 year, up to 2 years, 3 up to years, up to 4 years, or up to 5 years. For example, the composition may be stored in the dark for 1 hour to 90 days, from 24 hours to 60 days, from 2 days to 60 days, from 48 hours to 30 days, from 1 hour to 6 hours, from 6 hours to 24 hours, from 1 hour to 48 hours, from 6 hours to 72 hours, from 1 hour to 72 hours, from 12 hours to 1 week, from 2 hours to 48 hours, from 30 days to 90 days, from 60 days to 120 days, or from 6 hours to 2 weeks.

The composition for detecting BHB may be dry. The composition for detecting BHB may be dried by removing water or other solvent. As used herein, the term “dried” means that at least 90 wt %, 91 wt %, 92 wt %, 93 wt %, 94 wt %, 95 wt %, 96 wt %, 97 wt %, 98 wt %, 98.5 wt %, 99 wt %, 99.5 wt % or up to 100 wt % of water (or other solvent) in the composition for detecting BHB is removed. As used herein, the term “dry” means that the composition for detecting BHB has a water content of 10 wt % or less. For example, a dry composition for detecting BHB comprises less than 10 wt %, 9 wt %, 8 wt %, 7 wt %, less than 6 wt %, 5 wt %, 4 wt %, 3 wt %, 2 wt %, 1.5 wt %, 1 wt %, or less than 0.5 wt % water.

The composition for detecting BHB may further comprise an effective amount of a buffer for maintaining a desirable pH when the composition is exposed to an aqueous sample.

Buffers: Buffers as are known in the art may be used to maintain the pH of the composition for detecting BHB at a desired level, such as 8.5 or above, when the composition for detecting BHB is exposed to an aqueous sample. The composition for detecting BHB may be in the form of an aqueous solution or suspension. The composition for detecting BHB may comprise an effective amount of a buffer for maintaining a pH above 8.5 when the composition is in the form of an aqueous solution or suspension.

The amount of buffer that may be included may depend on the type of buffer employed and the desired pH. A non-limiting example of suitable buffer is “Tris”, tris(hydroxymethyl)aminomethane in combination with NaOH in an amount effective to render the pH of the composition for detecting BHB at 8.5 or above. Other non-limiting examples of suitable buffers include tricine, hydrazine, glycyglycine, EPPS (4-(2-Hydroxyethyl)-1-piperazinepropanesulfonic acid), HEPPS (4-(2-Hydroxyethyl)-1-piperazinepropanesulfonic acid), BICINE (N,N-Bis(2-hydroxyethyl)glycine), TAPS ([tris(hydroxymethyl)methylamino]propanesulfonic acid), and 2-amino-2-methyl-1,3-propanediol]. The pH of the composition for detecting BHB may be maintained at a basic pH, such as from 7.5 to 9.5, from 7.1 to 9, from 7.1 to 10, from 7.1 to 9, from 7.5 to 9, from 8 to 10, from 7.5 to 9.5, from 8 to 10, from 8 to 9, from 8.2 to 10, from 8.1 to 9.5, or from 8.5 to 9.5.

Aqueous samples: Non limiting examples of aqueous samples that may contain BHB to be detected by the composition for detecting BHB may be biological fluids such as blood, urine, obtained from a subject in need of testing for BHB. These biological samples may be diluted such as by adding water or a buffer.

The composition for detecting BHB may be on a carrier.

Carriers:

The composition for detecting BHB as disclosed herein may be on a carrier. The purpose may be to form a test strip, such as are known and used in the art, that may conveniently be dipped into or otherwise exposed to a biological fluid that is suspected to comprise BHB. If the composition for detecting BHB on the carrier undergoes a change in an optical property, for example changes from a light color to a dark color, then a user of the test strip would know that the biological sample contains BHB.

Such a carrier as disclosed herein, may be a porous material, such as filter paper, for example, Whatman filter paper #54 or similar. The composition for detecting BHB may be in the form of an aqueous (or other solvent) solution or suspension which may be applied to the porous carrier material, such as filter paper. The treated carrier material may dried to remove the water (or other solvent). As used herein, “dried” means that at least 90 wt %, at least 91 wt %, at least 92 wt %, at least 93 wt %, at least 94 wt %, at least 95 wt %, at least 96 wt %, at least 97 wt %, at least 98 wt %, at least 98.5 wt %, at least 99 wt %, at least 99.5 wt %, or up to 100 wt % of water in the composition for detecting BHB is removed. In other words, the composition for detecting BHB may comprise less than 10 wt %, less than 9 wt %, less than 8 wt %, less than 7 wt %, less than 6 wt %, less than 5 wt %, less than 4 wt %, less than 3 wt %, less than 2 wt %, less than 1.5 wt %, less than 1 wt %, or less than 0.5 wt % water after being dried.

In one embodiment, the carrier may further comprise an inert water-resistant substrate attached to a porous material. The inert water-resistant substrate may render the carrier easier to handle. For example, the composition on the carrier would be exposed to BHB in urine after the carrier is dipped into the urine.

Inert Water-Resistant Substrates:

Non-limiting examples of such inert water-resistant substrates include plastic sheets such as polyethylene, polypropylene, acrylics, polyesters, polycarbonate, polyvinylchloride, polystyrene and a combination thereof. The carrier material may be attached to the inert water-resistant substrate by, for example, gluing, or stapling, or taping.

A method of preparing a composition for detecting β-hydroxybutyrate (BHB) is provided. The method comprises the following steps:

(a) mixing nitro tetrazolium blue (NZT) or 2,3,5-triphenyltetrazolium chloride; β-hydroxybutyrate dehydrogenase (BHBD); diaphorase; and a redox cofactor to make a mixture, and

(b) adding to the mixture an inhibitor to make a composition having an optical property that changes upon exposure to BHB. A change in the optical property of the composition is reduced before the composition is exposed to the BHB. The redox cofactor is 2,3-dimethoxy-5-methyl-p-benzoquinone (coenzyme Q₀) or nicotinamide dinucleotide (NAD).

For each preparation method according to the present invention, a composition for detecting BHB is provided.

A method for detecting β-hydroxybutyrate (BHB) in a biological fluid from a subject is disclosed. The method may comprise the following steps: (a) exposing the biological fluid to a composition for detecting BHB according to the present invention such that the optical property of the composition is changed; and (b) detecting the change of the optical property in step (a). The detected change indicates the presence of BHB in the biological fluid.

The detection method may further comprise storing the composition for detecting BHB before step (a). The composition for detecting BHB may be stored for a predetermined time period, for example, at least 24 hours. The composition may be stored in the dark. The composition may be stored at a predetermined temperature of, for example, between 20° C. and 30° C. The biological fluid may be urine. The BHB may be present in the biological fluid in an amount above a predetermined level of BHB.

Levels of BHB in a biological fluid: According to the present invention, the level of BHB in the biological fluid may range from 0 mM to 4.0 mM. The amount of BHB in the biological fluid may be from 0.05 mM to 8.0 mM. The amount of BHB in the biological fluid may be from 0.1 mM to 7 mM, from 0.25 mM to 6 mM, from 0.2 mM to 5 mM, from 0.05 mM to 4.0 mM, from 2.0 mM to 4 mM, from 0.5 mM to 5 mM, from 1 mM to 5 mM, from 0.3 mM to 4 mM, from 1.5 mM to 5.5 mM, from 0.3 mM to 4 mM, from 1 mM to 6.0 mM, from 1.0 mM to 3.0 mM, limits inclusive.

Other Additives:

The composition for detecting BHB may further comprise other additives as are known in the art. Non-limiting examples of such additives include surfactants, such as nonionic and anionic surfactants.

### EXAMPLES

### General Test Procedures:

ΔE Measurement:

According to the invention as disclosed herein, β-hydroxybutyrate (BHB) may be detected in a biological fluid by observing an optical change in a composition for detecting BHB. As used herein, the term, optical change may mean a change in a spectrophotometric property, such as ΔE, that may be measured with a color meter. These instruments are known in the art, and generally use a color sensor, for example a photodiode together with on-board or separate software to produce a reading in the CIELAB color space which are the three coordinates: L, a and b, (also referred to as CIE L*a*b*), as defined by the International Commission on Illumination. The L coordinate relates to how light or dark the color is, the a coordinate relates to how red or green the color is, and the b coordinate relates to how blue or yellow the color is. Every color therefore has a unique L*a*b* coordinate. The ΔE values as used herein defined as the difference in the color, measured according the CIELAB color space between a reference, which is a carrier material that does not carry the composition for detecting BHB as disclosed herein, and a carrier material that does carry the composition for detecting BHB as disclosed herein. ΔE may be calculated according to the following formula:

ΔE=√[(Lᵣ−Lₘ)²+(aᵣ−aₘ)²+(bᵣ−bₘ)²]

Where: Lᵣ, aᵣ, and bᵣ are the L*a*b* coordinates of the reference color and Lₘ, aₘ, and bₘ are the L*a*b* coordinates of the measurement color.

### Preparation of Test Strips:

To make a urine test strip that is intended to test for the presence of β-hydroxybutyrate (BHB) in urine, it is necessary to first prepare a reaction mixture of the reagents to be applied to the test strips. Preparation of the reaction mixture is done according to the following procedure. All reagents in each composition for detecting BHB except for the BHBD and diaphorase were mixed ultrasonically at room temperature (approximately 25° C. to approximately 30° C.), and then stored in a dark room for 20 minutes at room temperature. In the darkroom, the BHBD and diaphorase are then added to form the reaction mixture that is applied to the test strips.

By micropipette, 0.5 microliters of the composition for detecting BHB were then dosed to a 6 mm×6 mm filter paper pad and dried in the dark (in a darkroom) for one hour or for 20 minutes at 40° C. When initially dosed with the composition for detecting BHB, the color of each pad was observed to be white to a pale yellow. It is important to retain a light pad color to provide a strong contrast in color once the test strips are dosed with BHB or human urine containing BHB, i.e. the completed test strips, are dosed with BHB or β-hydroxybutyric acid. Note that in the Examples section that β-hydroxybutyric acid is added to urine and a person having skill in the art will understand that the β-hydroxybutyric acid is a form of BHB, and accordingly, “BHB” in the Examples and throughout this disclosure is understood to refer to any form of β-hydroxybutyrate and therefore also encompasses β-hydroxybutyric acid. Each pad was then affixed with 3M 9969 Diagnostic Microfluidic Adhesive Transfer Medical Tape or Hi-Tech Products HT-187 pressure sensitive adhesive. to a 6 mm×75 mm strip of inert, waterproof material such as polyvinylchloride (PVC), polystyrene, Mylar®, polyester, polycarbonate, acrylic, or vinyl chloride/acetate, for example, to form a test strip.

### Determination of Test Strip Color Stability:

The following procedure was used to determine the relative efficacy of each inhibitor to reduce premature color change of the test strips. Each reaction mixture was dosed on an 18×18 mm pad formed from #54 Whatman filter paper using 9 doses of 5 microliter of the reaction mixture spaced equally across the large pad. For testing purposes the 18×18 mm pad was placed in a polycarbonate or polystyrene Petri dish for testing. The nine doses were all duplicates. The dosing was done at room temperature, approximately 25° C. (20° C.-30° C.) and the large pads were stored in the dark. In order to exclude as much light as possible, the samples were covered in aluminum foil and stored in an opaque cardboard or plastic box. A spectral colorimeter (BYK ColorGuard 2000 Colorimeter by Byk-Gardner, or NIX Pro Color Sensor by Nix Sensor Ltd., or CR-400 Chroma Meter by Konica Minolta) was used to measure the three-dimensional L*a*b* color coordinates of the dosed pad as it changes with time when stored at room temperature in the dark. The L*a*b* color measurements were converted to ΔE to provide a single measurement of color change. The L*a*b* color coordinates and ΔE were measured on a blank undosed filter paper as a control and then compared to the dosed samples. High ΔE values after storage represent a large color change from the control and are undesirable. For maximum visible color change during diagnostic testing with synthetic urine/BHB or human urine/BHB dosing it is desirable that test strips change color as little as possible in storage to maximize color contrast for BHB indication.

While instrumentation is useful to determine color change with or without BHB dosing, the main reason for a measurable, visual color change is so that instrumentation and equipment is not needed to measure a dosed strip. Accordingly, the subject can discern a visual difference on a dosed strip indicating BHB presence. Visual, subjective ratings of color change differences of a dosed strip as reported herein follow the guideline: 0=no color change; 1=pink to purple; 2=light purple; 3=medium purple; 4=purple; 5=dark purple.

Measurement of Relative Color Change of Test Strips when Exposed to 1-Hydroxybutyric Acid in Synthetic Urine

When measuring color change with BHB dosing, testing was done on 6 mm×6 mm squares of filter paper that are glued onto a 75 mm vinyl chloride/acetate support. When measuring color stability or color change over time without BHB dosing, testing was done on 18×18 mm squares of filter paper.

Freshly prepared or aged in the dark samples were then dosed with 0.2, 2.0 or 4.0 mM of β-hydroxybutyric acid in synthetic urine to simulate urine excretions of β-hydroxybutyrate (BHB). As soon as the samples were dosed, a timer was started to determine 3 minutes passage of time, at which point the samples were rated for color change compared to the change of the comparative examples, as noted in the following tables.

### Example 1 (Comparative): Control Sample with No Inhibitor

The following reagents shown in Table 1 were used for the Example 1 (comparative) control reaction mixture. Note that this mixture is similar that disclosed in U.S. Pat. No. 6,762,035.

TABLE 1
Example 1 (comparative), control composition for detecting
BHB (no inhibitor), NAD as redox coagent

| | Amount | | Wt % | Wt % |
|---|---|---|---|---|
| Ingredient | added | | (aqueous) | (dry) |
| 0.1M Tris/NaOH | 9.66 gm | | 96.5 | 25.1 |
| neutralized to | (0.117 gm dry) | | | |
| pH 8.7) | | | | |
| NAD (Sigma Aldrich) | 0.3 | gm | 3.0 | 64.3 |
| NZT (Sigma Aldrich) | 0.02 | gm | 0.2 | 4.3 |
| Diaphorase | 300 U | | 0.07 | 1.4 |
| (Worthington or | (total = 0.0066 g | | | |
| Sigma Aldrich) | | | | |
| BHBD (Creative | 2000 U | | 0.08 | 1.7 |
| Enzymes) | total = 0.008 g | | | |
| | BHBD | | | |
| Magnesium Chloride | 0.01 | gm | 0.1 | 2.2 |
| Surfynol ® 104 | 0.005 | gm | 0.05 | 1.1 |
| surfactant (nonionic | | | | |
| surfactant from | | | | |
| Evonik) | | | | |

NZT is nitro tetrazolium blue, IUPAC name 2-[2-methoxy-4-[3-methoxy-4-[3-(4-nitrophenyl)-5-phenyltetrazol-2-ium-2-yl]phenyl]phenyl]-3-(4-nitrophenyl)-5-phenyltetrazol-2-ium, usually used as the chloride salt, also referred to as Nitro blue tetrazolium chloride.

BHBD is β-hydroxybutyrate dehydrogenase

Tris is tris(hydroxymethyl)aminomethane, used as a buffer in conjunction with NaOH to produce the pH of 8.7.

As described below in Examples a), 1b), 1c), 1d), 1e), and 1f), various inhibitors were added to this Example 1 control reaction mixture to determine the efficacy of the various inhibitors at inhibiting color change of the composition for detecting BHB prior to exposure of the composition for detecting BHB to a sample comprising BHB.

### Example 1a) Effectiveness of Nanoparticles as Inhibitors

The following samples as shown in Table 1 were prepared and applied to 18×18 mm samples of filter paper as described above to test the efficacy of each nanoparticle to inhibit the color change of the pads after 60 hours in the dark, which was in an opaque dark box lined and covered with foil, in a darkroom controlled at 22° C. and 50% relative humidity. The results are shown in Table 2.

TABLE 2
Color stability of pads with added nanoparticles as inhibitors
to the reaction mixture, 60 hrs. dark, room temperature

| Sam- | | Amount of | | | | | % |
|---|---|---|---|---|---|---|---|
| ple | Added | inhibitor | | | | | change |
| ID | inhibitor | added¹ | L | a | b | ΔE | ΔE² |
| 521 | Example 1 | 0 | 78.8 | 3.5 | −0.6 | 11.6 | — |
| | Control | | | | | | |
| | (comparative) | | | | | | |
| 522 | Nano Anatase | 0.72% | 88.0 | −0.5 | 2.5 | 2.8 | −76 |
| | TiO₂ | | | | | | |
| | (invention) | | | | | | |
| 523 | Nano Rutile | 1.34% | 79.6 | 4.3 | −3.4 | 12.7 | +13 |
| | TiO₂ | | | | | | |
| | (invention) | | | | | | |
| 524 | Nano Zinc | 1.72% | 81.8 | 2.7 | −1.6 | 9.8 | −15 |
| | Oxide | | | | | | |
| | (invention) | | | | | | |
| 532 | Nano silica | 3.6% | 84.0 | 3.0 | 0.5 | 8.5 | −26 |
| | (invention) | | | | | | |
| 533 | +Nano CaCO₃ | 2.1% | 84.1 | 2.7 | −1.1 | 8.7 | −25 |
| | (invention) | | | | | | |
| 534 | +Nano | 1.6% | 85.8 | 1.7 | 0.5 | 6.4 | −45 |
| | Zirconium | | | | | | |
| | Oxide | | | | | | |
| | (invention) | | | | | | |

¹Amount of inhibitor added to reaction mixture in weight % of solid inhibitor on 0.5 g of liquid reaction mixture.
²Compared to the Example 1 control

The test strips as shown in Table 2 were then exposed to a sample of synthetic urine to which 0.2 mM of β-hydroxybutyric acid was added. The color change of the test strips, assessed visually as described above, relative to the control sample was shown as indicated.

TABLE 3
Colorimetric Response to BHB² in Urine

| | | Amount of | |
|---|---|---|---|
| | Added | inhibitor | Response with |
| ID | inhibitor | added¹ | 0.2 mM BHB |
| 521 | Example 1 Control | 0 | 4+ (purple/dark purple) |
| | (comparative) | | |
| 522 | +Nano Anatase TiO₂ | 0.72% | 3 (medium purple) |
| | (invention) (15-25 nm) | | |
| 523 | +Nano Rutile TiO₂ | 1.34% | 3+ (medium purple/purple) |
| | (invention) (5-30 nm) | | |
| 524 | +Nano Zinc Oxide | 1.72% | 3 (medium purple) |
| | (invention) (30-40 nm) | | |
| 532 | +Nano silica | 3.6% | 5 (dark purple) |
| | (invention) (5-35 nm) | | |
| 533 | +Nano CaCO₃ | 2.1% | 4 (purple) |
| | (invention) (50 nm) | | |
| 534 | +Nano Zirconium Oxide | 1.6% | 5 (dark purple) |
| | (invention) (45-55 nm) | | |

¹Amount of inhibitor added to reaction mixture in weight % of solid inhibitor on 0.5 g of liquid reaction mixture
²BHB = β-hydroxybutyric acid

The results shown in Tables 2 and 3 indicate that nanoparticles, in particular nanoparticulate titanium dioxide (anatase or rutile form), nanoparticulate silica and nanoparticulate zirconium oxide, can reduce premature color change of a test strip when the test strip is stored at room temperature (approximately 25° C.) in the dark for 60 hrs. It was surprising that the best nanoparticulate for inhibiting color change of the strip is nanoparticulate anatase titanium dioxide because nanoparticulate anatase titanium dioxide normally has very good UV blocking in the presence of light and the color change inhibition in this test occurs in the dark.

### Example 1b): Effectiveness of Sodium Nitrite, Calcium Nitrate and Two Levels of Hydroxyectoine as Inhibitors

The following samples as shown in Tables 4 and 5 were prepared and applied to 18×18 mm pads of filter paper as described above to test the efficacy of each additive to inhibit the color change of the samples after 60 hours in the dark, which was in an opaque dark box lined and covered with foil, in a darkroom controlled at 22° C. and 50% relative humidity. The results are shown in Table 4.

TABLE 4
Evaluation of sodium nitrite, calcium nitrate
and two levels of hydroxyectoine as inhibitors

| | | Amount of | ΔE after | |
|---|---|---|---|---|
| | Added | inhibitor | 60 Hrs. | % change |
| ID | inhibitor | added¹ | RT/Dark | ΔE² |
| 521 | Example 1 control | 0 | 11.6 | — |
| | (comparative) | | | |
| 540 | Sodium nitrite | 0.023 g | 7.8 | −15 |
| | (invention) | (4.6%) | | |
| 541 | Calcium nitrate | 0.027 g | 0.6 | −95 |
| | (invention) | (5.4%) | | |
| 542 | Hydroxyectoine | 200 mM; 0.0013 g | 2.1 | −82 |
| | (invention) | (0.27%) | | |
| 543 | Hydroxyectoine | 50 mM; 0.00033 g | 3.1 | −73 |
| | (invention) | (0.068%) | | |

The samples were also exposed to synthetic urine having 4.0 mM of added β-hydroxybutyric acid and the color change of each test strip was observed. The results are shown in Table 5.

TABLE 5
Colorimetric Response to 4.0 mM BHB² in synthetic urine

| ID | Description¹ | Amount | Response with 4.0 mM BHB |
|---|---|---|---|
| 521 | Example 1 Control | 0 | 4+ (purple/dark purple) |
| | (comparative) | | |
| 540 | Sodium nitrite | 0.023 g | 4+ (purple/dark purple) |
| | (invention) | (4.6%) | |
| 541 | Calcium nitrate | 0.027 g | 2 (light purple) |
| | (invention) | (5.4%) | |
| 542 | Hydroxyectoine | 200 mM | 2+ (light purple/medium purple) |
| | (invention) | 0.0013 g | |
| | | (0.27%) | |
| 543 | Hydroxyectoine | 50 mM | 2+ (light purple/medium purple) |
| | (invention) | 0.00033 g | |
| | | (0.068%) | |

¹Amount of inhibitor added to reaction mixture in weight percent of solid inhibitor on 0.5 g of liquid reaction mixture
²BHB = β-hydroxybutyric acid

The results in Tables 4 and 5 indicate that sodium nitrite, calcium nitrate and hydroxyectoine can inhibit premature color change of a test strip using NAD as the redox cofactor when stored at room temperature in the dark for 60 hours and still indicate the presence of BHB colorimetrically.

### Example 1c): Effectiveness of Various Levels of Trehalose as Inhibitors

Various levels of trehalose were added to the reaction mixture to evaluate the effectiveness of trehalose as an inhibitor and to determine if the trehalose was detrimental to the desired color change of the strips when exposed to a sample of urine with added β-hydroxybutyric acid. The results are shown in Tables 6 and 7.

TABLE 6
Color Stability of Test Strips with Trehalose stored
for 60 Hrs, room temperature in the dark.

| | | Amount of | ΔE after | |
|---|---|---|---|---|
| | Added | inhibitor | 60 Hrs. | % change |
| ID | inhibitor | added¹ | RT/Dark | ΔE² |
| 521 | Example 1 | 0 | 11.6 | — |
| (comparative) | Control | | | |
| 527 | Trehalose | 0.5M 0.0062 g | 2.4 | −79 |
| (invention) | | (1.24%) | | |
| 528 | Trehalose | 1M 0.0124 g | 2.3 | −80 |
| (invention) | | (2.48%) | | |
| 529 | Trehalose | 2M 0.025 g | 2.6 | −78 |
| (invention) | | (5.96%) | | |
| 530 | Trehalose | 3M 0.0374 g | 3.1 | −73 |
| (invention) | | (8.44%) | | |

¹Amount of inhibitor added to reaction mixture in grams of solid inhibitor on 0.5 g of liquid reaction mixture. Weight percent of solid inhibitor on 0.5 grams reaction mixture is in parenthesis
²Compared to the Example 1 control

The samples containing trehalose as the inhibitor were each exposed to synthetic urine with 4.0 mM of added β-hydroxybutyric acid and the color change was observed. The results are shown in Table 7.

TABLE 7
Colorimetric Response of Trehalose Strips to BHB/Urine²

| | Added | Amount of | |
|---|---|---|---|
| ID | inhibitor | inhibitor added¹ | Response with 4.0 mM BHB |
| 521 | Example 1 | 0 | 3 (medium purple) |
| | Control | | |
| | comparative | | |
| 527 | Trehalose | 0.5M; 0.0062 g | 3+ (medium purple/purple) |
| | (invention) | (1.24%) | |
| 528 | Trehalose | 1M; 0.0124 g | 3+ (medium purple/purple) |
| | (invention) | (2.48%) | |
| 529 | Trehalose | 2M; 0.025 g | 3 (medium purple) |
| | (invention) | (5.96%) | |
| 530 | Trehalose | 3M; 0.0374 g | 2+ (light purple/medium purple) |
| | (invention) | (8.44%) | |

¹Amount of inhibitor added to reaction mixture in grams of solid inhibitor on 0.5 g of liquid reaction mixture. Weight percent of solid inhibitor on 0.5 g reaction mixture in parenthesis.
²BHB = β-hydroxybutyric acid in synthetic urine

The results in Tables 6 and 7 indicate that trehalose can reduce premature color change of a test strip when stored at room temperature in the dark for 60 hours when NAD is used as the redox cofactor.

### Example 1d): Effectiveness of Magnesium Chloride and Barium Chloride as Inhibitors

Since a very low level of MgCl₂ (0.0001 g) was added to the control reaction mixture in Example 1 as shown above, the following experiments were conducted to determine if higher levels of MgCl₂ could act as an inhibitor to the undesired color change in the dark, but not inhibit the desired reaction to effect a color change when exposed β-hydroxybutyric acid in synthetic urine. Thus, a sample of the reaction mixture as above was prepared, but without any MgCl₂. In addition, samples were prepared in which MgCl₂ and BaCl₂ were added to the reaction mixture to evaluate the effectiveness of MgCl₂ and BaCl₂ as inhibitors and to determine if the MgCl₂ and BaCl₂ are detrimental to the desired color change of the strips when exposed to urine with added β-hydroxybutyric acid. The test strips were also exposed to synthetic urine containing 2.0 mM of β-hydroxybutyric acid. The results are shown in Tables 8 and 9.

TABLE 8
Color Stability with Magnesium chloride
compared to Barium chloride

| | | | ΔE after | % | ΔE after | % |
|---|---|---|---|---|---|---|
| | | Amount | 24 Hrs | change | 72 Hrs | change |
| | Added | inhibitor | RT/ | ΔE² | RT, | ΔE² |
| ID | inhibitor | added¹ | Dark | (24 hr) | Dark | (72 hr) |
| 601 | MgCl₂ | 0.0001 g | 6.9 | — | 13.8 | — |
| | (comparative) | (0.02%) | | | | |
| 602 | Example 1 | 0 | 8.1 | +17 | 15.9 | +15 |
| | (comparative) | | | | | |
| | control | | | | | |
| 603A | BaCL₂ | 0.007 g | 4.4 | −36 | 7.6 | −45 |
| | (invention) | (1.4%) | | | | |
| 603B | MgCL₂ | 0.007 g | 4.5 | −35 | 6.1 | −56 |
| | (invention) | (1.4%) | | | | |

¹Amount of inhibitor added to reaction mixture in grams of solid inhibitor on 0.5 g of liquid reaction mixture. Weight percent of solid inhibitor on 0.5 grams of reaction mixture is in parenthesis.
²Compared to the Example 1 control

TABLE 9
Colorimetric Response to 2.0 mM BHB² in synthetic Urine

| | Added | Amount of | |
|---|---|---|---|
| ID | inhibitor | inhibitor added¹ | Response |
| 601 | (Control) MgCl₂ | 0.0001 g (0.02%) | 4 (purple) |
| (comparative) | | | |
| 602 | Control without | 0 | 5 (dark purple) |
| (comparative) | very low level | | |
| | of MgCl₂ | | |
| 603A | BaCL₂ | 0.007 g (1.4%) | 4 (purple) |
| (invention) | | | |
| 603B | MgCL₂ | 0.007 g (1.4%) | 4 (purple) |
| (invention) | | | |

¹Amount of inhibitor added to reaction mixture, g of solid inhibitor on 0.5 g of liquid reaction mixture. Weight percent solid inhibitor on 0.5 g reaction mixture in parenthesis.
²BHB = β-hydroxybutyric acid in synthetic urine

The results in Tables 8 and 9 show that levels of barium chloride and magnesium chloride seventy times higher than the amount of magnesium chloride in the Example 1 control are effective at reducing the color change of the test strip when stored in the dark at room temperature, but do not interfere with the desired color change when the sample is exposed to β-hydroxybutyric acid in synthetic urine.

### Example 1e) Effect of Various Levels of Nanoparticulate Anatase Titanium Dioxide as Inhibitor

In this example, various levels of nanoparticulate anatase titanium dioxide were added to the Example 1 control reaction mixture to determine if the level of the nanoparticulate TiO₂ had inhibited the premature color change of the test strips or pads, without interfering with the desired color change when the strips (pads) were exposed to 2.0 mM β-hydroxybutyric acid in synthetic urine. The results are shown in Tables 10 and 11.

TABLE 10
Color Stability with Various amounts
of Nanoparticulate Anatase TiO₂

| | | | ΔE after | % change |
|---|---|---|---|---|
| ID | Inhibitor | Amount¹ | 72 Hrs RT, Dark | ΔE² |
| 601 | Example 1 | — | 13.8 | — |
| | (comparative) | | | |
| 613A | Nano Anatase TiO₂ | 0.33% | 13.4 | −3 |
| | (invention) | | | |
| 613B | Nano Anatase TiO₂ | 0.67% | 10.8 | −22 |
| | (invention) | | | |
| 613C | Nano Anatase TiO₂ | 1.34% | 5.2 | −62 |
| | (invention) | | | |

¹Amount of inhibitor added to reaction mixture in weight percent of solid inhibitor on 0.5 g of liquid reaction mixture
²Compared to the Example 1 control

TABLE 11
Colorimetric Response to 2.0 mM BHB² in synthetic Urine

| | | Amount¹ | |
|---|---|---|---|
| | | of | |
| ID | Inhibitor | Inhibitor | Response |
| 601 | Example 1 | 0 | 4 (purple) |
| (comparative) | | | |
| 613A | Nano Anatase | 0.33% | 2 (light purple) |
| (invention) | TiO₂ | | |
| 613B | Nano Anatase | 0.67% | 3+ (medium purple/purple) |
| (invention) | TiO₂ | | |
| 613C | Nano Anatase | 1.34% | 3 (medium purple) |
| (invention) | TiO₂ | | |

¹Added to example in Table 2, based on weight percent of solid inhibitor on 0.5 g of liquid reaction mixture.
²BHB = β-hydroxybutyric acid

The results summarized in Tables 10 and 11 show the minimum level of nanoparticulate anatase titanium dioxide necessary to reduce the color change of the test strip when stored in the dark for 72 hours at room temperature.

### Example 1f): Effect of Various Levels of Hydroxyectoine or Calcium Nitrate, or Nanoparticulate Anatase TiO₂ as Inhibitors with a Surfactant

In this example, various levels of hydroxyectoine, or calcium nitrate, or nanoparticulate anatase TiO₂ with a surfactant (Surfynol® 104) were added to the reaction mixture to determine if these materials inhibited the premature color change of the test strips or pads during storage, without interfering with the desired color change when the strips (pads) were exposed to 4.0 mM β-hydroxybutyric acid in synthetic urine. The results are shown in Tables 12 and 13.

TABLE 12
Color Stability with Added Nano Particles as Inhibitors to Reaction
Mixture, After 48 hours in the dark at 20° C.-30° C.

| | | | | % |
|---|---|---|---|---|
| | | Inhibitor | | change |
| ID | Inhibitor | Amount¹ | ΔE | ΔE² |
| 660 | Control (comparative) | 0 | 12.8 | — |
| 661 | Hydroxyectoine (invention) | 0.0015 g | 13.6 | +6 |
| | | (0.01%) | | |
| 662 | Hydroxyectoine (invention) | 0.0006 g | 14.5 | +13 |
| | | (0.004%) | | |
| 663 | Calcium Nitrate (invention) | 0.0027 g | 6.3 | −51 |
| | | (0.54%) | | |
| 664 | Nano Anatase TiO₂ + Surfactant | 1.34% Nano | 6.9 | −46 |
| | (invention) | Anatase | | |
| | | (0.002%) | | |
| | | surfactant | | |

¹Added to Example 1 control reaction mixture, grams of solid inhibitor on 0.5 g of liquid reaction mixture. Weight percent of solid inhibitor on 0.5 g reaction mixture is in parenthesis.
²Compared to the Example 1 control

The results shown in Table 12 demonstrate that calcium nitrate and nanoparticulate anatase titanium dioxide are effective at reducing a color change of the composition for detecting BHB (without BHB dosing) relative to Example 1 when stored at room temperature in the dark for 48 hours.

TABLE 13
Colorimetric Response to 4.0 mM BHB² in synthetic urine.

| | | Amount¹ | |
|---|---|---|---|
| | | Inhibitor | |
| ID | Inhibitor | added | Response |
| 660 | Example 1 Control | 0 | 4 (purple) |
| | (comparative) | | |
| 661 | Hydroxyectoine | 0.0015 g | 3 (medium purple) |
| | (invention) | (0.01%) | |
| 662 | Hydroxyectoine | 0.0006 g | 4 (purple) |
| | (invention) | (0.004%) | |
| 663 | Calcium Nitrate | 0.0027 g | 3 (medium purple) |
| | (invention) | (0.54%) | |
| 664 | Nano Anatase TiO₂ + | 1.34% Nano | 2 (light purple) |
| | Surfactant (invention) | Anatase | |
| | | (0.002%) | |
| | | surfactant | |

¹Added to Example 1 reaction mixture, grams of solid inhibitor on 0.5 g of liquid reaction mixture. Weight percent of solid inhibitor on 0.5 g reaction mixture is in parenthesis.
²BHB = β-hydroxybutyric acid

Table 13 demonstrates that that calcium nitrate and nanoparticulate anatase TiO₂ when added as inhibitors of a premature color change provide a colorimetric indication with BHB dosing though lighter in color than example 1.

### Example 2: Coenzyme Q₀ as Redox Cofactor to Detect β-Hydroxybutyrate in Urine

The following reaction mixture was used in Tables 14 and 15 as the Example 2 Control reaction mixture.

TABLE 14
Example 2, control composition for detecting BHB (no
inhibitor), Coenzyme Q₀ as redox coagent (invention)

| | Amount | | Wt % | Wt % |
|---|---|---|---|---|
| Ingredient | added | | (aqueous) | (dry) |
| 0.1M Tris/NaOH | 9.66 gm | | 98.9 | 52.1 |
| neutralized to pH 8.7 | (0.117 gm dry) | | | |
| Coenzyme Q₀ | 0.05 | g | 0.51 | 22.24 |
| β-cyclodextrin | 0.0082 | g | 0.084 | 3.6 |
| NZT | 0.02 | | 0.210 | 8.9 |
| Diaphorase | 300 Units | | 0.068 | 2.93 |
| (Worthington or | 0.0066 g | | | |
| Sigma Aldrich) | | | | |
| BHBD (Creative | 2000 Units | | 0.082 | 3.56 |
| Enzymes) | 0.008 g | | | |
| Magnesium Chloride | 0.01 | gm | 0.102 | 4.45 |
| Surfynol ® 104 | 0.005 | g | 0.051 | 2.22 |
| surfactant (nonionic | | | | |
| surfactant from | | | | |
| Evonik) | | | | |

The coenzyme Q₀ was premixed with the β-cyclodextrin. Without wishing to be bound by theory, it appeared that mixing the coenzyme Q₀ with the β-cyclodextrin according to the following procedure, enhanced greatly the solubility of the coenzyme Q₀ in water or in the buffered solution. As described below at Example 3, this procedure appears ineffective to enhance the solubility of coenzyme Q₁₀.

Preparation of Coenzyme Q₀ with β-Cyclodextrin:

First, 0.107 grams of β-cyclodextrin was dissolved in 4.8 grams of the Tris/NaOH buffer solution (pH 8.7). This mixture this heated at 45° C. for 5 minutes and then 0.082 g of coenzyme Q₀ was added. Heating was continued until coenzyme Q₀ was no longer visible as a solid. The coenzyme Q₀/β-cyclodextrin solution was used within 2 hours of making it. Alternatively, 0.0074 grams of coenzyme Q₀ and 0.0481 grams of β-cyclodextrin was mixed with 1.2070 grams water. This mixture was heated at 50° C. for 20 minutes with stirring. This produced a transparent solution with no visible particles. This procedure results in a fully solubilized form of coenzyme Q₀ which is optically transparent and has no visible particles of coenzyme Q₀ in the mixture. It is important that the coenzyme Q₀ mixture is fully soluble so that it may be mixed with other water-soluble ingredients and applied to a paper test strip for further reactions with biological solutes. If the coenzyme Q₀/β-cyclodextrin mixture is not water soluble, it cannot provide a uniform distribution of reactions for the test for BHB in a biological fluid as disclosed herein.

Then, the Example 2 test strips were prepared as described above for the Example 1 test strips and stored for 24 at room temperature in the dark. As in Example 1, the test strips were stored in a light free box, covered with aluminum foil at a temperature between 20° C. and 30° C. for the indicated periods of time. The ΔE values were measured after storage for 24 hours and are shown below in Table 15.

TABLE 15
Color Stability of Test Strips with coenzyme Q₀ with
Various inhibitors after storage in the dark for
24 hours between 20° C. and 30° C.

| | | Inhibitor | |
|---|---|---|---|
| ID | Inhibitor | Amount¹ | ΔE² |
| 660 (comparative) | Example 1 Control (NAD) | 0 | 12.8 |
| 666 (invention) | Example 2 Control | 0 | 12.7 |
| | (Coenzyme Q₀) | | |
| 667 (invention) | 666 + crosslinker³ | 0.01% | 19.9 |
| 668 (invention) | 666 + Nanoparticulate | 1.34% | 4.9 |
| | Anatase TiO₂ | | |
| 669 (invention) | 666 + Trehalose | 5.96% | 22.0 |

¹Added to Control Example 2 reaction mixture, based on percent actives of total reaction mixture. This is weight percent of solid inhibitor on 0.5 g reaction mixture.
²Versus Example 1 Control
³Polyfunctional azridine crosslinker (Crosslinker ® CX-100, DSM)

The results shown in Table 15 demonstrate that coenzyme Q₀ in place of NAD for the redox cofactor, combined with nanoparticulate anatase TiO₂ exhibits much less color change, measured as ΔE, in a test strip stored in the dark at room temperature for 24 hours.

Next, test strip samples prepared as described above were exposed to 2.0 mM β-hydroxybutyric acid in synthetic urine and the color of the strips was then recorded. The results are shown in Table 16.

TABLE 16
Colorimetric Response to 2.0 mM BHB² in synthetic urine

| | | Amount¹ | |
|---|---|---|---|
| | | Inhibitor | |
| ID | Inhibitor | added | Response |
| 660 | Example 1 | 0 | 4+ (purple/dark purple) |
| (comparative) | Control (NAD) | | |
| 666 | Example 2 | 0 | 5 (dark purple) |
| (invention) | Control | | |
| | (coenzyme Q₀) | | |
| 667 | 666 + 0.01% | 0.01% | 1 (very light purple) |
| (invention) | crosslinker³ | | |
| 668 | 666 + Nano | 1.34% | 0.5 (pinkish purple) |
| (invention) | Anatase TiO₂ | | |
| 669 | 666 + Trehalose | 5.96% | 4 (purple) |
| (invention) | | | |

¹Amount added to Example 2 control reaction mixture, weight percent of solid inhibitor on 0.5 g reaction mixture
²BHB = β-hydroxybutyric acid, synthetic urine from Carolina Supply.
³polyfunctional azridine crosslinker (Crosslinker ® CX-100, DSM)

The results shown in Table 16 shows that coenzyme Q₀ is more effective than NAD as a redox cofactor in terms of intensity of a color change of the composition for detecting BHB when the composition for detecting BHB is exposed to BHB.

### Example 3 Comparative: Solubility of Coenzyme Q₁₀ and β-Cyclodextrin in Water

Example 2, above, demonstrates how heating β-cyclodextrin and coenzyme Q₀ in a buffered base results in a clear solution. The same procedure as disclosed above was repeated with β-cyclodextrin and coenzyme Q₁₀ and the coenzyme Q₁₀ could not be adequately incorporated in the basic buffer solution. The procedure for solubilizing coenzyme Q1₀ in U.S. Patent Application Publication No. US 2007/0202090 A1 was followed and results are listed below in Table 17. Coenzyme Q₁₀ and β-cyclodextrin were combined with water in the following molar ratios of coenzyme Q₁₀ to β-cyclodextrin: 1:1; 1:5; and 1:10. Each solution was heated for 5 hours at 75° C. and then an additional 5 hours at 85° C. The results, in terms of the appearance of the resulting mixtures are tabulated below in Table 17.

TABLE 17
Appearance of coenzyme Q₁₀ and β-cyclodextrin mixtures in water after
heating for 5 hours at 75° C. and 5 hours at 85° C.

| Molar Ratio | 1:1 | 1:5 | 1:10 |
|---|---|---|---|
| coenzyme | 0.2000 | 0.0510 | 0.0436 |
| Q₁₀ (grams) | | | |
| β-cyclodextrin | 0.3005 | 0.3000 | 0.6075 |
| (grams) | | | |
| water | 2.6000 | 3.0000 | 3.0000 |
| (grams) | | | |
| Appearance | not transparent; | not transparent; | not transparent; |
| | visible particles | visible particles | visible particles |
| | | and sediment | and sediment |

Upon cooling the samples shown in Table 17, the undissolved contents of the mixtures settled to the bottom of each container. A portion of this precipitated solid from each sample was removed and then mixed with a large portion of water. These diluted samples each formed a turbid dispersion of particulates that later precipitated to the bottom of each container indicating that the solids were not water soluble.

Therefore, the mixtures of coenzyme Q₁₀ with β-cyclodextrin do not appear to form a solution in water, but instead appear to form a dispersion of particles which is unlikely to provide a uniform mixture for reactivity when exposed to BHB in a biological fluid, particularly such a fluid comprising water, e.g., urine. As shown in Table 16, the procedures detailed in U.S. Patent Application Publication No. US 2007/0202090 A1 do not appear to adequately disperse, or dissolve coenzyme Q₁₀ in an aqueous solution. This data clearly demonstrates that coenzyme Q₁₀ is unlikely to be effective as a redox cofactor in an aqueous reaction, such as would occur when the solution for detecting BHB is exposed to a biological fluid such as urine, for example.

In some embodiments, the invention herein can be construed as excluding any element or process that does not materially affect the basic and novel characteristics of the composition or process. Additionally, in some embodiments, the invention can be construed as excluding any element or process not specified herein.

As noted previously, although the invention is illustrated and described herein with reference to specific embodiments, the invention is not intended to be limited to the details shown. Rather, various modifications may be made in the details within the scope and range of equivalents of the claims and without departing from the invention.

Within this specification, embodiments have been described in a way which enables a clear and concise specification to be written, but it is intended and will be appreciated that embodiments may be variously combined or separated without departing from the invention. For example, it will be appreciated that all preferred features described herein are applicable to all aspects of the invention described herein.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

What is claimed is:

1. A composition for detecting β-hydroxybutyrate (BHB), wherein the composition has an optical property that changes upon exposure to BHB, the composition comprising:
(a) nitro tetrazolium blue (NZT) or 2,3,5-triphenyltetrazolium chloride,
(b) β-hydroxybutyrate dehydrogenase (BHBD),
(c) diaphorase, and
(d) a redox cofactor, wherein the redox cofactor is 2,3-dimethoxy-5-methyl-p-benzoquinone (coenzyme Q₀).

2. A composition for detecting β-hydroxybutyrate (BHB), wherein the composition has an optical property that changes upon exposure to BHB, comprising:
(a) nitro tetrazolium blue (NZT) or 2,3,5-triphenyltetrazolium chloride,
(b) β-hydroxybutyrate dehydrogenase (BHBD),
(c) diaphorase,
(d) a redox cofactor, wherein the redox cofactor is 2,3-dimethoxy-5-methyl-p-benzoquinone (coenzyme Q₀) or nicotinamide dinucleotide (NAD), and
(e) an inhibitor in an amount effective for reducing a change in the optical property of the composition for at least 6 hours before the composition is exposed to the BHB.

3. The composition of claim 2, wherein the redox cofactor is coenzyme Q₀ and the inhibitor is selected from the group consisting of nanoparticulate anatase TiO₂, nanoparticulate ZnO, nanoparticulate silica, nanoparticulate CaCO₃, nanoparticulate ZrO₂, NaNO₂, Ca(N₃)₂, hydroxyectoine, and calcium nitrate.

4. The composition of claim 2, wherein the redox cofactor is coenzyme Q₀ and the inhibitor is nanoparticulate anatase TiO₂.

5. The composition of claim 2, wherein the redox cofactor is NAD and the inhibitor is selected from the group consisting of nanoparticulate ZnO, nanoparticulate ZrO₂, NaNO₂, Ca(NO₃)₂, and trehalose.

6. The composition of claim 1, wherein the redox cofactor is coenzyme Q₀ and the BHB is in an aqueous sample, the composition further comprising cyclodextrin in an amount effective for solubilizing the coenzyme Q₀ in the aqueous sample.

7. The composition of claim 6, wherein the aqueous sample is a biological fluid.

8. The composition of claim 7, wherein the biological fluid is urine.

9. The composition of claim 1, wherein the composition has a water content less than 0.3 wt %.

10. The composition of claim 1, wherein the composition further comprises an effective amount of a buffer for maintaining a pH above 8.5 when the composition is exposed to an aqueous sample.

11. The composition of claim 1, wherein the composition is on a carrier.

12. The composition of claim 11, wherein the carrier comprises a porous material.

13. The composition of claim 12, wherein the carrier further comprises an inert water-resistant substrate attached to the porous material.

14. A method of preparing a composition for detecting β-hydroxybutyrate (BHB), comprising:
(a) mixing nitro tetrazolium blue (NZT) or 2,3,5-triphenyltetrazolium chloride; β-hydroxybutyrate dehydrogenase (BHBD); diaphorase; and a redox cofactor to make a mixture, wherein the redox cofactor is 2,3-dimethoxy-5-methyl-p-benzoquinone (coenzyme Q₀) or nicotinamide dinucleotide (NAD), and
(b) adding to the mixture an inhibitor to make a composition having an optical property that changes upon exposure to BHB, wherein a change in the optical property of the composition is reduced for at least 6 hours before the composition is exposed to the BHB.

15. A method for detecting β-hydroxybutyrate (BHB) in a biological fluid from a subject, comprising:
(a) exposing the biological fluid to the composition of claim 1 or a composition prepared according to the method of
(i) mixing nitro tetrazolium blue (NZT) or 2,3,5-triphenyltetrazolium chloride; β-hydroxybutyrate dehydrogenase (BHBD); diaphorase; and a redox cofactor to make a mixture, wherein the redox cofactor is 2,3-dimethoxy-5-methyl-p-benzoquinone (coenzyme Q₀) or nicotinamide dinucleotide (NAD), and
(ii) adding to the mixture an inhibitor to make a composition having an optical property that changes upon exposure to BHB, wherein a change in the optical property of the composition is reduced for at least 6 hours before the composition is exposed to the BHB, whereby the optical property of the composition is changed; and
(b) detecting the change of the optical property in step (a), wherein the detected change indicates the presence of BHB in the biological fluid.

16. The method of claim 15, further comprising storing the composition for at least at least 24 hours before step (a).

17. The method of claim 15, wherein the biological fluid is urine.

18. The method of claim 15, wherein the BHB is present in the biological fluid in an amount of at least 0.2 mM.

19. The method of claim 15, wherein the BHB is present in the biological fluid in an amount of from 0.2 mM to 4 mM.

20. The method of claim 16, wherein the biological fluid is urine
